(19) United States
(12) Patent Application Publication
(10) Pub. No.: US 2024/0156734 A1
(43) Pub. Date: May 16, 2024

(54) Simplified Method for Making Shelf Stable Highly Water Soluble Anhydrous Compositions Of Cyclodextrin Complexes of Macrocyclic Lactones

(71) Applicant: Michael Farber, Livingston, NJ (US)

(72) Inventor: Michael Farber, Livingston, NJ (US)

(21) Appl. No.: 18/218,312

(22) Filed: Jul. 5, 2023

Related U.S. Application Data
(63) Continuation-in-part of application No. 17/985,338, filed on Nov. 11, 2022, now abandoned.

(51) Int. Cl. A61K 9/14; A61K 9/16

(52) U.S. Cl. CPC A61K 9/145; A61K 9/146; A61K 9/1682

(57) ABSTRACT

A shelf stable highly water soluble anhydrous powder composition of complexed macrocyclic lactones, formed by dissolving a macrocyclic lactone, a cyclodextrin, and a polysorbate compound in an organic solvent in specific ratios as outlined herein. The resultant organic solution is dried under oxygen and moisture free conditions to form an anhydrous powder composition.

### FIELD OF THE DISCLOSED TECHNOLOGY

The present invention is directed towards an anhydrous powder composition comprising a macrocyclic lactone, a polysorbate, and a cyclodextrin carrier, wherein the macrocyclic lactone is complexed with a cyclodextrin carrier in an organic solvent under anhydrous conditions.

### BACKGROUND

Polysorbates (PS) are nonionic, amphipathic surfactants commonly used in the pharmaceutical and food industry. They are widely used as emulsifiers or stabilizers of protein in pharmaceutical compositions, more specifically biopharmaceutical compositions comprising proteins, antibodies, enzyme etc. polysorbates are oily liquids derived from ethoxylated sorbitan esterified with fatty acids. polysorbate 20 to polysorbate 80 (Polyoxyethylene (20) sorbitan monooleate) are commonly used in the industry. polysorbates, depending upon the hydrophobic tail of the compound, may have varying degrees of solubility in water. In particular, polysorbate 80 has been shown to advantageously interfere with the aggregation of large proteins in solution and therefore is widely used as a surfactant/stabilizer in the biopharmaceutical industry. Polysorbate 80 has been shown to effectively increase the solubility of macrocyclic lactones in solution, but the macrocyclic lactones are highly susceptible to both hydrolysis and oxidative degradation in solution when combined with polysorbates. Therefore a completely shelf stable, highly soluble composition of these compounds is still lacking.

Despite the wide usage of polysorbates in the biopharmaceutical industry, certain disadvantages have been associated with the use of polysorbates. polysorbates are easily hydrolyzed by residual host proteins, mainly enzymes. These enzymes have been identified in the prior art mainly as lipases including phospholipase and carboxylesterase. During hydrolysis, the fatty acid ester bond present in polysorbates is cleaved to release and free fatty acid. It is often difficult to prevent the hydrolysis of the polysorbate as the enzymes involved in hydrolysis are difficult to knock-out due to their very low concentrations in the host cell proteins. polysorbates 20 and 80 have been shown to undergo hydrolysis in solution releasing peroxides and other oxidative moieties with an estimated half-life of 6 months at 30 degrees Celsius. This in turn affects the drug potency, destabilizes the pharmaceutical formulation and reduces the shelf-life of the pharmaceutical composition and the active ingredient.

Another disadvantage associated with polysorbates is that they undergo auto-oxidation by light, temperature, or by transition trace metals. The peroxide formed during the auto-oxidation reaction can lead to oxidation of active ingredients of the pharmaceutical composition. Both polysorbate 20 and 80 release an initial burst of peroxide in solution with polysorbate 80 being more labile. This destabilizes the composition and leads to loss in activity of the active agent.

Alkyl polyoxyethylene side chains of polysorbates undergo auto-oxidation resulting in formation of hydroperoxides, side-chain cleavage, and eventually formation of short chain acids such as formic acid—all of which can affect the stability of the product. It has been discovered that these oxidant moieties and peroxides can have a negative effect upon the shelf life of the molecules such as proteins, vaccines, or macrocyclic lactones in solution. Oxidative degradation of the protein molecule in a biopharmaceutical composition leads to loss of drug potency over time and may challenge formulation development and reduce shelf life. Oxidation is one of the major chemical degradation pathways for protein pharmaceuticals, causing the adverse effect on drug potency and shelf life. The observed protein oxidation may involve photo degradation due the presence of peroxide in the commercially available polysorbates. Photooxidation, especially the oxidation of light sensitive amino acids such as Tryptophan, Tyrosine and Phenylalanine present in the proteins, may lead to increased physical instability and aggregation of the protein. Quality of surfactants such as polysorbate 80 can also significantly influence the photostability of a protein or stability of labile compounds in solution. It has been widely reported that the commercially available polysorbates are a diverse mixture and the expected structure of the polysorbates account only for 20% of the total polysorbates.

In addition, polysorbate 80 has a disadvantage of taking as long as over one hour to fully dissolve in water at room temperature. The problems associated with use of polysorbates as protein stabilizing agents have been reviewed by many authors. Various workers have tried to suggest use of a different concentration of the polysorbates to avoid the problem associated with the aggregation/oxidation of protein due to the presence of hydrogen peroxide in the commercially available polysorbates. Agarkhed et al, 2013 discloses that polysorbate 80 increased the susceptibility of monoclonal antibodies to oxidation at a concentration of $0.10% of polysorbates due to an increase in methionine oxidation. Authors observed that oxidation of three methionine residues at M111, M251, and M427 (all in heavy chain), one moderate change at M357 (in heavy chain), and one at Tryptophan 32 (in light chain) contributed to protein oxidation. Authors also report that Tryptophan may play a significant role in photodegradation pathways of proteins even though it is present in relatively low amounts in proteins when compared to other amino acids. It was concluded by the authors that higher oxidation of amino acids present in the monoclonal antibodies at higher polysorbate 80 concentrations due to the presence of higher amount of peroxide in polysorbate 80 caused an increase in oxidation of Tryptophan and Methionine residues after light exposure.

Similarly Zhou et. al., 2021 evaluated the effect of polysorbate 80 on the self-assembly mechanism, structure, morphology and stability of insulin amyloid-like spherulites. The amyloid spherulites are amyloid fibrils are arranged into large ordered spheroid structures, known as spherulites and are characterized by a spherical symmetry consisting of a central dense core from which a low-density corona grows radially with a radius extending up to few μm to mm. The human insulin has a tendency towards aggregation and also formation of aggregates other than fibrils, i.e. amyloid spherulites. Authors in this particular study observed that polysorbate 80 induced a delay in the onset of amyloid spherulites formation but did not completely prevent aggregation of insulin. Amyloid spherulites with a higher content of intermolecular beta-sheets in the presence of the surfactant above its critical micelle concentration was observed, in addition, to a denser packing leading to a more stable aggregate.

In order to avoid hydrolysis and oxidation of the polysorbate, even the addition of antioxidants to these solutions cannot adequately extend the half-life. Furthermore, in the case of injectable solutions of pharmaceutical or medicinal molecules, only a limited number of antioxidants are approved by the Food and Drug Administration (FDA) for such use. Therefore, there remains a need for a composition or a method that enables the use of polysorbate as a solubilizer and emulsifier while overcoming the known drawbacks of the polysorbate in an aqueous solution for long term storage of the proteins or molecules degraded by oxidative processes or oxides especially in a biopharmaceutical composition.

The studies in the prior art shows that polysorbates can have a solubilizing effect on cyclodextrin complexes of macrocyclic lactones but these solutions can not be fully stabilized. Even the use of inert gas purging and refrigeration can not completely eliminate hydrolysis of the macrocyclic lactones solutions of the prior art thus formed thus curtailing shelf life and dramatically increasing costs.

A simple cost-effective solution to the problem of both oxidative and hydrolytic degradation of cyclodextrin complexes of macrocyclic lactones in solution is to use a “one step” organic solvent based method to produce the final anhydrous composition comprising or containing all the required elements in the proper ratios. Thus the polysorbates can be effectively used to increase solubility and bioavailability of the complexed macrocyclic lactones while their anhydrous compositions will have 100% stability and long shelf life. The resultant anhydrous compositions can be advantageously used as top dressing in animal feeds, compressed into micro or regular pellets, and/or used in hard or soft chews whether alone or in combination with other anti-parasitic compounds for administering to pets or farm animals or used as a coating in various feeds. The compositions either alone, or in combination with other water soluble molecules or complexes, can be administered either orally as drench or put into drinking water where the solutions are used for a length of time between 1 and 8 hours from their confection so that degradation of any of the components is minimized and the efficacy of the compositions is maintained.

### SUMMARY OF THE INVENTION

In one aspect of the present invention, an anhydrous powder composition for pharmaceutical products includes a macrocyclic lactone, a polysorbate compound, and a cyclodextrin, wherein the macrocyclic lactone is first dissolved in an organic solvent such as pure methanol. Sufficient cyclodextrin, such as 2 hydroxypropyl beta cyclodextrin, is added to this solution so that the macrocyclic lactone is fully complexed in the organic solution. A sufficient polysorbate compound such as polysorbate 80 is then added to this organic solution, so that the final macrocyclic lactone anhydrous composition, when added to water, can achieve a solution that is between 3% and 4% macrocyclic lactone. The solution is 3% macrocyclic when used with ivermectin and 4% when used with moxidectin. Sufficient cyclodextrin is then added to this organic solution, so that the ratio of polysorbate to total cyclodextrin in the composition is minimally 1:5 by weight. The polysorbate compound may be selected from the group consisting of polysorbate 20, polysorbate 40, polysorbate 60 and polysorbate 80. The cyclodextrin carrier may be selected from the group consisting of alpha cycylodextrin, beta cycylodextrin, gamma cycylodextrin and/or at least one of an alpha-, beta- and gamma-cyclodextrin derivative. A weight ratio of polysorbate to total cyclodextrin in the composition is in the range from 1:5 to 1:10 such as 1:5.

In an embodiment, the polysorbate compound is polysorbate 80 and the cyclodextrin carrier is 2 hydroxypropyl beta cyclodextrin, wherein a ratio of polysorbate 80 to total hydroxypropyl beta cyclodextrin in the composition is minimally 1:5.

In an embodiment, the composition has substantially zero or equivalent moisture content and is manufactured in a moisture free environment using dry nitrogen or any dry inert gas to prevent oxidation of the components

In an embodiment, the polysorbate compound is devoid of peroxide content and moisture. The organic solvent is pure and completely devoid of moisture in embodiments of the disclosed technology. The manufacturing method Is devoid of moisture and oxygen.

In an embodiment, the macrocyclic lactone composition contains ivermectin and the polysorbate compound is either polysorbate 20 or 80 preferably polysorbate 80 In an embodiment, the pharmaceutical composition includes an antioxidant selected from the group consisting of C-tochopherol, tochopherol acetate, butylated hydroxytoluene (BHT), ascorbic acid, tocopherol, propyl gallate and mixtures thereof, the antioxidant present in an amount ranging from about 0.05% to about 3% by weight of the composition. In an embodiment, the pharmaceutical composition is in a dosage form of either powders, mini or micro pellets, troches, oral drenches, water solubilized liquid feed, injections or compressed tablets, coated tablets, soft or hard chewables, or simple top dressings. In an embodiment, the compressed tablets are enteric coated. Further, in another embodiment, the pellet troches can be coated with a long chain fatty acid with oil based flavor having a melting point greater than 50 degrees Celsius. The pharmaceutical composition when being tableted can further include a lubricating agent, wherein the lubricating agent is selected from the group consisting of anhydrous carboxymethylcellulose, hydroxyethylcellulose, hydroxypropyl methylcellulose, hydroxypropyl cellulose, or a mixture thereof and, wherein the lubricating agent is present in an amount of from about 0.05 to about 5% by weight of the composition. The tabletting components in embodiments of the disclosed technology are moisture free and the tablet and coating formation is done in an oxygen free, moisture free environment.

Other features and advantages of the present instant invention will be apparent from the detailed description, and from the appended claims. Thus, other aspects of the present instant invention are described in the following disclosure and are within the ambit of the present instant invention.

### DESCRIPTION OF EMBODIMENTS OF THE DISCLOSED TECHNOLOGY

In the following description, certain specific details are set forth in order to provide a thorough understanding of various embodiments of the present instant invention. However, one skilled in the art will understand that the invention is not limited to the embodiments described herein, and are not intended to represent the scale of the various embodiments. It should be understood that the detailed description is not intended to limit the invention to the particular form disclosed, but on the contrary, the invention is to cover all modifications, equivalents, and alternatives falling within the scope of the present instant invention as defined by the appended claims. As used throughout this description, the word “may” is used in a permissive sense (i.e. meaning having the potential to), rather than the mandatory sense, (i.e. meaning must). Further, the words “a” or “an” mean “exactly one” and the word “plurality” means “two or more” unless otherwise mentioned.

In this specification, whenever a composition or an element or a group of elements is preceded with the transitional phrase “comprising”, it is understood that we also contemplate the same composition, element or group of elements with transitional phrases “consisting of”, “consisting”, “selected from the group of consisting of, “including”, or “is” preceding the recitation of the composition, element or group of elements and vice versa.

Unless the context requires otherwise, throughout the present specification and claims, the word “comprise” and variation thereof, such as, “comprises” and “comprising” are to be construed in an open, inclusive sense that is as “including, but not limited to”.

Reference throughout this specification to “one embodiment” or “an embodiment” means that a particular feature, structure or characteristic described in connection with the embodiment is included in at least one embodiment of the present instant invention. Thus, the appearances of the phrases “in one embodiment” in various places throughout this specification are not necessarily all referring to the same embodiment. Furthermore, the particular features, structures, or characteristics may be combined in any suitable manner

The term “polysorbates” refers to amphipathic, nonionic surfactants. polysorbates (PS) are made from ethoxylated sorbitan or isosorbide, a sorbitol derivative that has been esterified with fatty acids. polysorbates, notably polysorbatepolysorbate 20 (PS20) and polysorbatepolysorbate 80 (PS80), are most often employed surfactants in biopharmaceutical formulations to stop protein denaturation, aggregation, surface adsorption, and flocculant formation during thawing.

The term “polysorbate 20” commonly known as Tween 20 and Kotilen-20 refers to nonionic surfactant of the polysorbate type created by the ethoxylation of sorbitan monolaurate. The term “polysorbate 40” or “Tween 40”, also known as polyoxyethylene sorbitan monopalmitate, where 40 stands for monopalmitate, refers to a nonionic surfactant consisting of sorbitol, ethylene oxide, and palmitic acid. The molecular formula of polysorbate 40 is C62H122O26. The term “polysorbate 60” or “Tween 60” refers to polyoxyethylene sorbitan monostearate, consisting of sorbitol, ethylene oxide, and stearic acid. The molecular formula of polysorbate 60 is C64H126O26. The term “polysorbate 80” or “Tween 80” refers to sorbitan mono-9-octadecenoate poly(oxy-1,2-ethanediyl) derivatives and are complex mixtures of polyoxyethylene ethers. The molecular formula of polysorbate 80 is C64-H124-O26.

The term “Cyclodextrin” refers to cyclic oligosaccharides, which consist of a macrocyclic ring of glucose subunits joined by α-1,4 glycosidic bonds. Cyclodextrins are synthesized from starch by enzymatic conversion. They are used in food, pharmaceutical, drug delivery, and chemical industries, as well as agriculture and environmental engineering. Cyclodextrins possess a number of glucose monomers ranging from six to eight units in a ring, creating a cone shape:

The term “α-1,4 glycosidic bonds α (alpha)-cyclodextrin” refers to cyclic oligosaccharides, that consists of a macrocyclic ring of glucose subunits joined by α-1,4 glycosidic bonds having six glucose subunits. The term “β (beta)-cyclodextrin” refers to cyclic oligosaccharides, which consist of a macrocyclic ring of glucose subunits joined by α-1,4 glycosidic bonds having seven glucose subunits. The term “γ (gamma)-cyclodextrin” refers to cyclic oligosaccharides that consist of a macrocyclic ring of glucose subunits joined by α-1,4 glycosidic bonds having 8 glucose subunits.

The ability of the pharmaceutical dosage form to preserve the physical, chemical, therapeutic, and microbiological qualities while being stored and used by the patient is referred to as “drug stability”. This ability is determined by the rate at which the pharmacological dose forms vary. Drug stability has an impact on the product's safety and effectiveness; degrading impurities may result in a loss of potency and produce potential negative consequences. In order to guarantee the quality and safety of pharmaceuticals, it is crucial to achieve chemical and physical stability.

The invention generally relates to an anhydrous powder composition, comprising a macrocyclic lactone, a cyclodextrin and polysorbate. The polysorbate is devoid of moisture and peroxide content. The aforementioned are combined with a cyclodextrin, wherein the final anhydrous composition is formed by adding the ingredients in specific ratios to an organic solvent that can effectively dissolve all components, mixing the first components of macrocyclic lactone and cyclodextrin in a ratio which will allow complete complexion in solution. Then the polysorbate is added to the organic solution and fully dissolved, after which excess cyclodextrin is added to the organic solution such that the final ratio by weight of polysorbate to total cyclodextrin in the organic solution is minimally 1:5.

Moreover, the anhydrous powder composition contains polysorbate that is selected from a group comprising polysorbate 20, polysorbate 40, polysorbate 60 and polysorbate 80. Furthermore, the anhydrous powder composition also contains cyclodextrin, which may be selected from a group comprising alpha cyclodextrin, beta cyclodextrin, and gamma cyclodextrin, wherein the cyclodextrin may be a derivative of an the alpha-, beta-, or gamma-cyclodextrin. The cyclodextrin, or cyclodextrin derivative, used in embodiments of the disclosed technology is highly water soluble and also highly soluble in the organic solvent. An advantage of polysorbate is the increased solubility of the macrocyclic lactone complexes in water based solutions while the anhydrous composition substantially avoids degradation in such solutions over time.

polysorbates (PS) are amphipathic, nonionic surfactants made from ethoxylated sorbitan or isosorbide, a sorbitol derivative that has been esterified with fatty acids. The most often employed surfactants in biopharmaceutical formulations to stop protein denaturation, aggregation, surface adsorption, and flocculant formation during thawing are polysorbates, notably polysorbate 20 (PS20) and polysorbate 80 (PS80).

Cyclodextrins possess a hydrophobic interior and a hydrophilic exterior that helps to create complexes with hydrophobic compounds. The Cyclodextrins including Alpha, beta-, and gamma-cyclodextrin have been applied for delivery of a variety of drugs, including hydrocortisone, prostaglandin, nitroglycerin, itraconazol, and chloramphenicol. The cyclodextrin confers solubility and stability to these drugs. The inclusion compounds of cyclodextrin with hydrophobic molecules are able to penetrate body tissues that may be used to release biologically active compounds under specific conditions.

The α-cyclodextrin is composed of six glucose subunits which are linked end to end via α-1,4 linkages. The arrangement of six primary alcohols on one face and twelve secondary alcohol groups on the other leads to a tapered cylinder shape with the exterior surface of cyclodextrins being hydrophilic whereas the interior core is hydrophobic. The α-cyclodextrin confers aqueous solubility to hydrophobic drugs and stability to labile drugs. β-Cyclodextrin is a cyclic oligosaccharide consisting of seven glucose subunits joined by α-(1,4) glycosidic bonds forming a truncated conical structure. It is widely used in food, pharmaceutical, cosmetics, and chemicals industries. Gamma-cyclodextrin possesses a larger cavity with respect to the β-cyclodextrin. The cavity may henceforth be covered with a wider range of guest molecules. Gamma-Cyclodextrin is a ring-shaped molecule made up of eight glucose units linked by alpha-1,4-bonds. The solubility degree of gamma-cyclodextrin in water is higher than alpha-cyclodextrin and beta-cyclodextrin, and it is used to allow formation of inclusion complexes with bigger molecules. Gamma-cyclodextrin may be used to produce inclusion compounds, which may be used to stabilize medicines, make deliquescing, sticky, and liquid medicine more powder-like, and/or make insoluble or hard-soluble medicine soluble.

In another embodiment of the present invention, the cyclodextrins may be chosen from any of the alpha, beta or gamma cyclodextrins or their derivatives that are compatible with the formulations and the route of administering, such that dissolution in a water medium is almost instantaneous (within 1 second, ½ second, or ⅛ second).

In various embodiments of the present invention, the compositions must be manufactured in a humidity free environment without moisture or heat in excess of 30 degrees Celsius for any prolonged period, so as to preserve the polysorbate and prevent the release of peroxide and oxidants and to prevent any hydrolysis by preventing any amount of moisture being present during the fabrication process of the composition.

Some of non-limiting examples of β-cyclodextrin which can be used in the composition of the present invention include 2-Hydroxypropyl-β-cyclodextrin (HP-β-CD), sulfobutylether β-cyclodextrin and randomly methylated-β-cyclodextrin (RM-β-CD).

In various embodiments of the present invention, additional antioxidants and glidants may be added to the compositions. In another embodiment of the present invention, the composition of the present invention is a methanolic solution after all components have been dissolved and then processed through a 20 micron filter to effect sterilization before drying under sterile conditions. The dried, sterile anhydrous composition is then held/placed in a single or plurality of sterile opaque vial(s) with nitrogen inert dried gas and stored at less than 30 degrees Celsius for use in injectable solutions (solutions which can be safely injected into an animal (which can be a human)).

When needed, the composition is then mixed with sterile water in the vial to the indicated volume in order to obtain an injectable solution of 3% macrocyclic lactone or less, as desired. The anhydrous composition in the vial, when stored properly, has a shelf life in excess of 2 years. When mixed with sterile water or other water based sterile injectable medium, in embodiments of the disclosed technology, the solution must be used within one hour and ideally kept away from light and heat to keep degradation to a minimum.

In yet another embodiment of the present invention, the composition of the present invention can be effectively used for production of highly soluble shelf stable complexes of the macrocyclic lactones including both avermectins and milbemycins.

According to another embodiment of the present invention, the composition comprises of an antioxidant in an amount (ranging from about 0.05% to about 3% by weight of the composition) effective to prevent the degradation of the macrocyclic lactones, wherein the anti-oxidant is selected from the group consisting of C-tochopherol, axtochopherol acetate, butylated hydroxytoluene (BHT), ascorbic acid, tocopherol, propyl gallate, and mixtures thereof.

In a further embodiment of the present invention, the anhydrous powder composition may be advantageously used as a coating composition. Absorption of any moisture may be prevented by combining the composition of the present invention with a long chain edible fatty acid with a melting point sufficiently above the maximum ambient storage temperature (such as stored in an ambient temperature less than 30 degrees Celsius) of the feed. By way of example, an edible long chain fatty acid combined with the anhydrous powder composition of the invention along with an appropriate amount of ultra fine hydrophobic-SiO2(H—SiO2) particles embedded in the fatty acids can be used to obtain a water-repelling, hydrophobic composition that allows coating of the anhydrous powder composition whilst protecting the composition from either external moisture once used in feeding or internal moisture of the feed pellets themselves during storage.

In one embodiment, ivermectin is first dissolved in pure methanol under anhydrous and oxygen free conditions. Sufficient moisture free hydroxypropyl-beta-cyclodextrin (or 2-hydroxypropyl-γ-cyclodextrin) is added to this methanolic solution in an amount sufficient to completely complex the ivermectin while in solution. After this methanolic solution has been stirred for sufficient time to ensure complete complexation, polysorbate 80, in a quality devoid of moisture and peroxide, is added to the methanolic solution in a quantity sufficient to obtain a water based 3% ivermectin solution of the final anhydrous composition. To this methanolic solution, Once all the above components are thoroughly dissolved, excess hydroxypropyl beta cyclodextrin is added in an amount where the ratio of the weight of polysorbate 80 in the solution to the weight of total hydroxypropyl beta cyclodextrin in the solution is minimally a ratio of 1:5. The methanolic solution is then dried under oxygen free and moisture free conditions in accordance with parameters known in the art for such production but not exceeding a maximum temperature where degradation occurs. This maximum temperature for drying is 30 C in embodiments of the disclosed technology, and further, this step can take place in the dark. The anhydrous ivermectin composition is, in some embodiments, formed in a vacuum (defined as less than 0.2 atmospheric pressure) at a temperature of 30 C or less to avoid degradation of the polysorbate, and then is sealed under a nitrogen blanket to prevent moisture and is stored as such, separate from light and/or moisture and excess heat. In some embodiments, the anhydrous composition is stored under a nitrogen blanket where the nitrogen is dried nitrogen devoid of all moisture.

In another embodiment of the present invention, the anhydrous powder composition may be advantageously combined with a feed additive for cattle/livestock under anhydrous conditions along with a coating to prevent moisture absorption from either external or internal sources. Addition of polysorbates to the feed of the livestock has been associated with significantly higher productivity in animals such as higher milk yield, increased rate of weight gain, higher efficiency in converting feed into body tissues or milk, and/or a reduction in manure production. In another embodiment, the composition can be added to the feed additive of a livestock at a concentration of 0.1 to 1% under anhydrous conditions along with a coating to prevent moisture absorption. Coatings such as edible long chain fatty acids are known in the art and can be selected for melting temperatures and moisture resistance desirable in each location. Water impermeability and resistance can be adjusted by addition of further additives to the edible coating as well as fat soluble additives such as vitamins and nutrients which can be added to the coatings if so desired.

In yet another embodiment, the anhydrous powder composition of the present invention may be combined with other anti parasitic compositions in chewable dog or cat treats. These chewables may be hard or soft chewables, as desired. They may contain only the desired macrocyclic lactone anhydrous composition such as ivermectin or moxidectin anhydrous composition of the invention. Alternatively, the compositions disclosed herein can be combined with other anti-parasitics as are known in the art, whether as simple molecules or complexes, thus providing chewables with broad spectrum anti-parasitic infection for both cats and dogs. These chewables may use anhydrous flavorings as are known in the art to be desired by cats and/or dogs.

In another embodiment, feed can be coated with the macrocyclic lactone anhydrous compositions of the invention by embedding same in edible long chain fatty acid or other edible water impermeable coatings such as are known in the art that are solid (do not melt at standard temperature and pressure). The feeds are stored at less than degrees Celsius in embodiments of the disclosed technology for this reason. These coatings can be, for example, made with edible long chain fatty acids into which the anhydrous powder compositions are mixed after melting of the fatty acids. These coatings can have further additives to enhance nutrition, taste, and water impermeability.

In still another aspect of the invention, the composition may be in a dosage form of powders, micro, mini, or regular pellets, troches, or compressed tablets. The compressed tablets may be enteric coated, wherein the pellet dosage forms may be coated with a long chain fatty acid with oil based flavor having a melting point at or above 50° C. The powder mixture of the composition of the present invention may be stored away from light and under a dry nitrogen blanket in sealed glass or aluminum containers and stored at preferably less than 30 C.

Pharmaceutical lubricants are the agents added to formulations in a very small quantity (usually 0.25%-5.0%, w/w) to improve the powder processing properties of formulations. Lubricants help to decrease friction at the interface between a tablet's surface and the die wall during ejection so that the wear on punches and dies are reduced. They prevent the sticking of tablets to punch faces as well as sticking of capsules to dosators and tamping pins. In terms of powder flow, lubricants may help to improve the flow of blends and aid unit operations.

In yet another embodiment of the present invention, when the compositions are used in tabletting, an effective amount of a lubricating agent is added to the anhydrous composition prior to tabletting, wherein the lubricating agent is selected from the group consisting of anhydrous carboxymethylcellulose, hydroxyethylcellulose, hydroxypropylmethylcellulose, hydroxypropylcellulose, and mixtures thereof, wherein the lubricating agent is present in an amount of from about 0.05 to about 5% by weight of the composition.

The foregoing discussion is simply illustrative of the invention. However, since many embodiments of the invention can be made without departing from the spirit and scope of the invention, the invention resides wholly in the claims hereinafter appended.

Any device or step to a method described in this disclosure can comprise or consist of that which it is a part of, or the parts which make up the device or step. The term “and/or” is inclusive of the items which it joins linguistically and each item by itself. “Substantially” is defined as “at least 95% of, at up to and including 100% of, the term being described” and any device or aspect of a device or method described herein can be read as “comprising” or “consisting” thereof.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalence of the claims are to be embraced within their scope. Combinations of any of the methods, systems, and devices described herein-above are also contemplated and within the scope of the disclosed technology.

What is claimed is:

1. A method of forming an anhydrous powder composition for pharmaceutical composition(s), comprising components of:
a macrocyclic lactone:
a polysorbate compound; and
a cyclodextrin;
in a substantially dried form produced as a single composition by drying of the mixture of said components which were previously dissolved together in an organic solvent;
wherein the polysorbate compound is polysorbate 80;
wherein the cyclodextrin carrier is hydroxypropyl beta cyclodextrin; and wherein a weight ratio of polysorbate 80 to hydroxypropyl beta cyclodextrin is at least 1:5.

2.-5. (canceled)

6. A composition made using the method of claim 1, wherein the composition and any of its components before addition to the organic solvent has substantially zero or equivalent moisture content and is manufactured in a moisture free environment using dry inert gas.

7. A composition made using the method of claim 1, wherein the polysorbate compound is devoid of peroxide content.

8. A composition made using the method of claim 1, wherein the organic solvent is substantially all, or completely methanol.

9. A composition made using the method of claim 1, wherein the macrocyclic lactone is ivermectin.

10. The composition according to claim 9, wherein the anhydrous ivermectin composition has a solubility in water of 20 to 30 mg/ml of ivermectin to water.

11. A composition made using the method of claim 1 wherein the macrocyclic lactone is moxidectin.

12. A composition made using the method of claim 11, wherein the anhydrous moxidectin composition has a solubility of 30 to 40 mg/ml of moxidectin in water.

13. A composition made using the method of claim 1 made by first dissolving the macrocylic lactone in an organic solvent.

14. The composition of claim 13, wherein hydroxypropyl beta cyclodextrin is required to fully complex the macrocylic lactone in solution.

15. The composition of claim 14, wherein sufficient polysorbate is added to a methanolic solution to achieve final solubility of the macrocyclic lactone of the anhydrous composition of 30 mg/ml for ivermectin in water.

16. The composition of claim 14, wherein sufficient polysorbate is added to the methanolic solution to achieve final solubility of the macrocyclic lactone of the anhydrous composition of 40 mg/ml for moxidectin in water.

17. A composition made using the method of claim 1, wherein coated products using the anhydrous composition are formed by mixing the anhydrous powder composition with a melted long chain fatty acid.

18. The according to claim 17, further comprising fatty acids having a melting point greater than 50 degrees Celsius.

19. The composition according to claim 9, further comprising a lubricating agent, wherein the lubricating agent is selected from one or a combination of anhydrous carboxymethylcellulose, hydroxyethylcellulose, hydroxypropyl methylcellulose, and hydroxypropyl cellulose; and
wherein the lubricating agent is present in an amount of from about 0.05 to about 5% by weight of the composition.

20. The composition according to claim 2, having a shelf life in excess of two years.

21. A method of forming an anhydrous powder composition for pharmaceutical composition(s), consisting essentially of:
a macrocyclic lactone:
a polysorbate compound; and
a cyclodextrin;
in a substantially dried form produced as a single composition by drying of the mixture of said components which were previously dissolved together in an organic solvent.

22. A method of forming an anhydrous powder composition for pharmaceutical composition(s), consists of:
a macrocyclic lactone:
a polysorbate compound; and
a cyclodextrin;
in a substantially dried form produced as a single composition by drying of the mixture of said components which were previously dissolved together in an organic solvent.
